(19) United States
(12) Patent Application Publication
(10) Pub. No.: US 2006/0147557 A1
(43) Pub. Date: Jul. 6, 2006

(54) Composition for treating amyloidoses and/or oxidation damage in nervous system

(75) Inventors: Rey-Yuh Wu, Taipei County (TW); Yau-Jan Chyan, Taipei County (TW); Tzung-Hsien Lai, Taipei County (TW)

Correspondence Address: LADAS & PARRY, 26 WEST 61ST STREET, NEW YORK, NY, 10023, US

(73) Assignee: DEVELOPMENT CENTER FOR BIOTECHNOLOGY

(21) Appl. No.: 11/030,623

(22) Filed: Jan. 6, 2005

(51) Int. Cl. A61K 36/704; A61K 36/899; A61K 36/48

(52) U.S. Cl. 424/725; 424/750; 424/757

(57) ABSTRACT

The present invention provides a composition for treating amyloidoses and/or oxidation damage in nervous system comprising a therapeutically effective amount of the water extract of polygonum multiflorum. A method for treating amyloidoses and/or oxidation damage in nervous system of a subject is also provided.

### BACKGROUND OF THE INVENTION

1. Field of the invention

The invention mainly relates to a herbal composition; more particularly, to a herbal composition for treating amyloidoses and/or oxidation damage in nervous system.

2. Description of the Related Art

In vivo, proteins fold and unfold constantly in order to get biologically compliable “three-dimensional assembly”. However, when a mistake which leads to unnatural conformation of protein occurs, it usually causes one disease of the family called amyloidoses, in which misfolded peptides accumulate in or around cells and form aggregates. Aggregates of misfolded proteins are implicated in various neurodegenerative diseases such as (1) Alzheimer disease which forms intracellular neurofibrillary tangles and extracellular amyloid plaques; (2) prion diseases where PrPSc aggregates; (3) Kennedy disease where intranuclear inclusions are abserved; (4) Pick disease which forms Tau inclusions; (5) Parkinson disease where Lewy bodies are highlighted; and (6) Machado-Joseph disease where mutant ataxin-3 forms intranuclear inclusions (Taylor J P et al., “Toxic proteins in neurodegenerative disease,” Science, 296: 1991-5, 2002; M. Bucciantini et al., “Inherent toxicity of aggregates implies a common mechanism for protein misfolding diseases,” Nature, 416:507-11, 2002; J. Xu et al., “Dopamine-dependent neurotoxicity of α-synuclein: A mechanism for selective neurodegeneration in Parkinson disease,” Nat Med, 8:600-6, 2002; R. Kayed et al., “Common structure of soluble amyloid oligomers implies common mechanism of pathogenesis,” Science, 300:486-9, 2003). Most symptoms associated with systemic amyloidoses are due to the physical build-up of these amyloid deposits in vital organs.

Furthermore, the structures of protein aggregate are similar. For example, the structure of extracellular plaque associated with Alzheimer disease is similar to those of intracellular protein aggregates observed in other amyloidoses, such as Parkinson disease, spinocerebellar ataxia, and prion diseases, and is also similar to that of intracellular tau clumps known as neurofilbrillary tangles in Alzheimer disease. In addition, Alzheimer and Parkinson diseases frequently coexist in clinic observations. Although different diseases relate to different proteins (e.g. Parkinson disease, spongiform encephalopathies and Alzheimer disease relate to synuclein, prion protein and amyloid, respectively), aggregates of the protein but not the fibrillar form are regarded as the real culprit (M. P. Lambert et al., “Diffusible, nonfibrillar ligands derived from Aβ1-42 are potent central nervous system neurotoxins” Proc Natl Acad Sci, 95:6448-53, 1998).

Alzheimer disease, for example, is a neurodegenerative disease, which damages the brain cortex and hippocampal to cause memory impairment, loss of language and visuospatial skills and behavior deficits. Alzheimer disease is characterized by the aggregates of amyloid beta (Aβ) peptide outside the cell and the neurofibrillary tangles of tau. Age is regarded as a main factor of Alzheimer disease. The incidence of the elder aged more than 65 is about 4%, and that of the elder aged more than 80 approaches 40%. Although it is a pity that the real mechanism and therapeutic strategy are not well established, mutations of the three genes, APP, PS1 and PS2, are proven to be the main factor of early onset familiar Alzheimer disease. In addition, apoE (apolipoprotein E) is highly related to late onset Alzheimer disease.

The main components of senile plaques of Alzheimer disease are 40 to 43 amino acids amyloid peptides. In the APP mutated animal model, the amyloid aggregate increases to form senile plaques as age increases and leads to neuron degeneration and death, behavior change and memory loss. The forming of senile plaque is in proportion to such symptoms. Although there are no direct clinical relations between plaques and clinical severity, mediating plaque formation to treat amyloidoses is suggested.

New compounds or agents for therapeutic regimes to inhibit or reverse amyloid formation, deposition, accumulation and/or persistence that occur(s) in Alzheimer disease and other amyloidoses are therefore desperately needed.

### SUMMARY OF THE INVENTION

The invention provides a composition for treating amyloidoses and/or oxidation damage in nervous system comprising a therapeutically effective amount of the water extract of polygonum multiflorum.

The invention also provides a method for treating amyloidoses and/or oxidation damage in nervous system of a subject comprising administrating the subject a therapeutically effective amount of the water extract of polygonum multiflorum.

### BRIEF DESCRIPTION OF THE DRAWINGS

FIG. 1 illustrates the effect of the water extract of polygonum multiflorum for inhibiting oxidation damage in SK—N—SH cells.

FIG. 2 illustrates the spatial working memory improving effect of the water extract of polygonum multiflorum in rats injected with Aβ40 in the brain.

FIG. 3 illustrates the spatial reference memory improving effect of the water extract of polygonum multiflorum in rats injected with Aβ40 in the brain.

FIG. 4 illustrates the spatial working memory improving effect of the water extract of polygonum multiflorum in Tg2576 mice and non-transgenic mice.

FIG. 5 illustrates the spatial reference memory improving effect of the water extract of polygonum multiflorum in Tg2576 mice and non-transgenic mice.

FIG. 6 illustrates the spatial working memory improving effect of the water extract of polygonum multiflorum in Tg2576 mice and non-transgenic mice.

FIG. 7 illustrates the spatial reference memory improving effect of the water extract of polygonum multiflorum in Tg2576 mice and non-transgenic mice.

FIG. 8 illustrates the body weight changes in the mice treated with the water extract of polygonum multiflorum.

### DETAILED DESCRIPTION OF THE INVENTION

The present invention mainly provides a composition for treating amyloidoses and/or oxidation damage in nervous system comprising a therapeutically effective amount of the water extract of polygonum multiflorum.

As used herein, the term “amyloidoses” refers to a disease or symptom that relates to abnormal peptide aggregate inside or outside the cell, especially in the nervous system. Such peptide aggregate in the nervous system usually causes degeneration of motor neuron and then leads to changes in multiple aspects such as memory, language, behavior, or mood. Preferably, the amyloidoses according to the invention comprise Alzheimer disease, stroke, dementia, prion diseases, spongiform encephalopathies, Kennedy disease, Pick disease, Parkinson disease, spinocerebellar ataxia, and Machado-Joseph disease. More preferably, the amyloidoses according to the invention comprise Alzheimer disease.

The composition according to the invention can be used for inhibiting neural cell death due to amyloid peptide aggregate. As a result, the composition according to the invention has anti-Aβ effect.

In the animal model of the invention, the composition is effective in improving memory of APP mutant and Aβ40 injected mice. It evidenced that the composition according to the invention is useful for inhibiting neuronal damage resulting from the amyloid β peptide aggregation in vivo, and preferably amyloid β 40 or 42 peptide aggregation. Therefore, the symptoms caused by abnormal peptide aggregation such as memory impairment, loss of language and visuospatial skills and behavior deficits are improved when treated with the composition according to the invention. In the animal model illustrated in the invention, the spatial working memory and spatial reference memory are both improved in the APP transgenic mice and Aβ 40 injected rats treated with the composition according to the invention.

Furthermore, the composition according to the invention is also used for treating oxidation damage in the nervous system. In the animal model of the invention, the composition inhibits cell death caused by oxidation damage of neural cell.

The active component of the composition according to the invention is the water extract of polygonum multiflorum. Polygonum multiflorum, also known as Ho-shou-wu, is traditionally used by the Chinese to promote longevity. This herb is rich in flavonoids and stilbenes is primarily used to restore vigor, strengthen the cardio-vascular system, enhance the endocrine system, treat daze, tone the liver and kidneys, increase physical energy, help to renew sexual potency and help to improve hair loss and hair color density. Polygonum multiflorum also has been employed as a remedy for insomnia, stomach upset, and diabetes. Furthermore, polygonum multiflorum is good in applications for chronic fatigue or degenerative conditions. The dramatic effect of the water extract of polygonum multiflorum in treating amyloidoses and/or oxidation damage is un-exceptedly found in the invention.

Not wishing to be bound by theory, it is believed that the actual mechanism of the water extract of polygonum multiflorum for treating amyloidoses and/or oxidation damage in nervous system may be: (1) the protein contents in the brain and liver are increased; (2) the malondialdehyde (MDA) contents in the brain and liver are decreased; (3) the superoxide dismutase (SOD) in the brain is elevated; (4) lipid peroxidation (LPO) is decreased; and (5) the activities of monoamine oxidase-B (MAO-B) in the brain and liver are lowered.

The water extract of polygonum multiflorum can be produced by many methods. Conventional methods of producing a herb extract is applicable to the invention. In one embodiment of the invention, polygonum multiflorum is extracted for ready use. In another embodiment of the invention, polygonum multiflorum is provided as a semi-manufacture and the extraction procedure is carried out by a user before use. In a preferred embodiment of the invention, the water extract is prepared by extracting polygonum multiflorum with water at a temperature of from 60 to 70° C. For convenient manipulation, polygonum multiflorum is pieced and then extracted. The extract can be further filtered to remove residues. In a more preferred embodiment of the invention, the water extract is further concentrated and dried to form powder to facilitate application.

In another aspect, polygonum multiflorum according to the invention is boiled with a black bean liquid and/or yellow rice wine as described in the traditional record. Polygonum multiflorum boiled with black-bean liquid and/or yellow rice wine according to a traditional process is called red fo ti. The treatment of polygonum multiflorum is regarded to eliminate the toxicity and enhance the curative effect.

According to the invention, the composition can be administrated in several forms. It may be consumed as a pharmaceutical composition or as a food composition. Preferably, the composition according to the invention is in the form of powder, tablet, capsule, solution, tonic, or food.

The invention also provides a method for treating amyloidoses and/or oxidation damage in nervous system of a subject comprising administrating the subject a therapeutically effective amount of water extract of polygonum multiflorum.

The following examples are given for the purpose of illustration only and are not intended to limit the scope of the present invention.

### EXAMPLE 1

### Water Extract of Polygonum Multiflorum

The grinded polygonum multiflorum with a weight of 20 g was refluxed in 10-folded of distilled water (60 to 70° C.) for 1 hour. The resulting suspension was filtered with a #100 filter and then centrifuged at 4° C./100,000 rpm for 30 minutes. The water-soluble fractions from the extractions were concentrated under reduced pressure and dried for obtaining power. The yield of polygonum multiflorum is about 30% and that of black bean boiled polygonum multiflorum is about 20%.

### EXAMPLE 2

### Water Extract of Polyponum Multiflorum for Inhibiting Aβ40 Toxicity In Vitro

Cell culture for neuroblastoma SK—N—SH and glioblastoma C6 cells: SK—N—SH and C6 cells were grown in the high glucose DMEM (Hyclone®, USA) containing 10% FBS (Biological Industriesg, Israel), 25 mM HEPES (GIBCO®, USA), and antimicrobial antibodies (GIBCO®, USA) at 37° C., 5% CO₂ incubator. Amyloid beta 25-35 peptide (Aβ25-35) and all the chemicals were obtained from Sigma®, unless specified. Aβ1-40 was purchased from Tocris® (MO, USA).

Cell viability assay-MTT reduction method: The SK—N—SH or C6 cells were cultured in a 96-well plate with a seeding density of about 10,000 cells (about 60% confluency). As the cell density reached 85-90% confluence, cells were treated with 100 μL of the condition media (DMEM containing 0.5% FCS and respective concentrations of polygonum multiflorum extract and 5 μM of Aβ25-35 or 1-40 peptide) for 24 hrs in 37° C., 5% CO₂ incubator for MTT reduction measurement. The MTT reduction assay was performed as described by Hansen et al. (J. Immu. Method (119), 203-210, 1989). Briefly, after 24-hr incubation, the neuronal cultures were further incubated with the MTT solution (final concentration of MTT is 0.5 mg/mL) for another 2 hrs at 37° C. The culture were then lysed overnight with the lysis buffer [50% N,N-dimethylformamide, 20% SDS and adjust the pH to 4.8-4.7 with 20% Acetic acid and 80% of 1N HCl] at 37° C. Cell viability was calculated as % of MTT reduction (measured at λ=570 nM) compared to the value obtained from the control group (neurons treated without peptide). The representative values were derived from an independent experiment of triplicate, expressed as means ±s.d.

The result shows for that polygonum multiflorum with the concentration of less than 0.1 μg/μL, the cell viability is 95 to 100%. It evidences that polygonum multiflorum has anti-Aβ1-40 effect on inhibiting Aβ40 toxicity.

### EXAMPLE 3

### Water Extract of Polygonum Multiflorum for Treating Oxidation Damage In Vitro

The method for assaying water extract of polygonum multiflorum on treating oxidation damage is similar to the method as described in Example 2 except the condition media containing hydrogen peroxide with various concentrations.

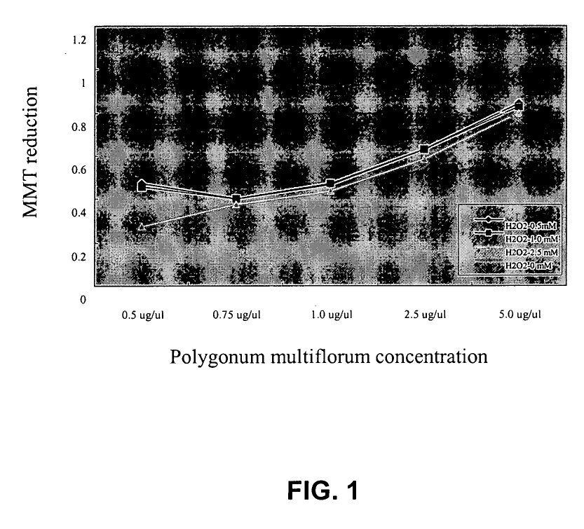

The result is shown in FIG. 1. It evidences that the water extract of polygonum multiflorum has the ability to treat oxidation damage and inhibit cell death.

### EXAMPLE 4

### Water Extract of Polygonum Multiflorum for Inhibiting Aβ40 Toxicity in Rats Injected with Amyloid in the Brain

To develop an animal model of Alzheimer disease, Aβ40 was infused into the rat cerebral ventricle using an osmotic pump. The performance caused neural damage to the hippocampus and behavior change of some memory tasks in the beta-amyloid protein-treated rats (1994 Nabeshima).

Alzet® osmotic pump was perfused with amyloid β-peptide 1-40 solution and assembled with the brain infusion kit for leading the amyloid β-peptide 1-40 solution full of the pump and the PE tube of the kit. The rats were anesthetized with 45 mg/kg sodium pentobarbital. The lateral ventricle of the anesthetized rat was positioned with a stereotaxic instrument. With the assistance of the stereotaxic instrument, Alzet® osmotic pump was disposed in the hypodermis of the back neck. The rats were sutured and grown in the cage. On days 8 to 9 after operation, passive avoidance response of the rats was evaluated and the rats were subjected to Morris water maze on days 10 to 15. In the control group, the pump was filled with 35% acetonitrile/0.1% trifluoacetic acid. In the experiment group, water extract of polygonum multiflorum was administrated daily and before passive avoidance response and/or Morris water maze evaluations.

The Morris water maze consisted of a round tank (pool) with a diameter of 160 cm, depth of 60 cm. The tank was filled with 23±1° C. water with a depth of 35 cm. The tank was divided equally into four quadrants by four points, which were designated as eastern, western, southern, and northern points. The quadrants were designated as northwest, southwest, northeast, and southeast. A circular rigid platform of a diameter of 15 cm was fixed on the southwest beneath the water level with 1 cm. The circumference of the tank was covered with a patterned screen that provided the clue of exploring of the rats. A CCD camera was disposed above the tank and connected to a computer for tracking by utilizing image capture.

The rats were subjected to the water maze test once a day for three serial days. There were four practical opportunities each day. The rats had 122 seconds to search for the platform locating at the southwestern quadrant each time. If the rat did not search for the right platform during limited time, it was guided to the captioned platform. The rats rested for 15 seconds after reaching the platform. All the processes, including the swimming paths and escaping time, were recorded by computerized tracking system. In view of approaching commonly reached during the third day to the fifth day, the platform was removed on the fourth day. Subsequently, the time difference between where the rats appear in the original location of platform and the other regions was observed. The data of duration and distance of the rat's searching the platform were evaluated with ANOVA analysis. The data of previous four tests were deemed as the performance of spatial working ability of the rats. Additionally, the data of the fourth day of test reflected the performance of spatial reference memory ability of the rats. On the fifth day of test, the rats had four training opportunities to search new location of the platform, and on the fourth hour after testing, the rats were trained once again, so that the working memory tests were obtained.

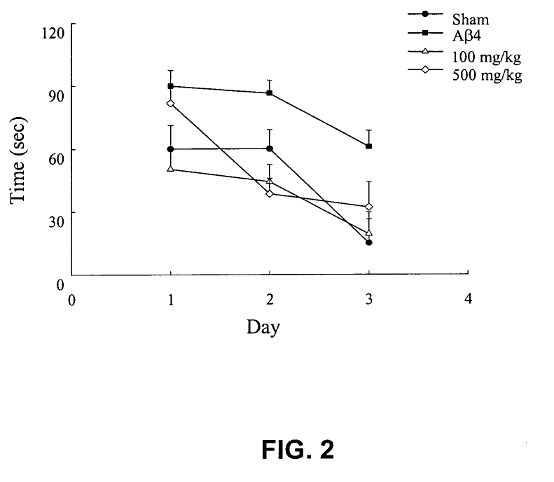

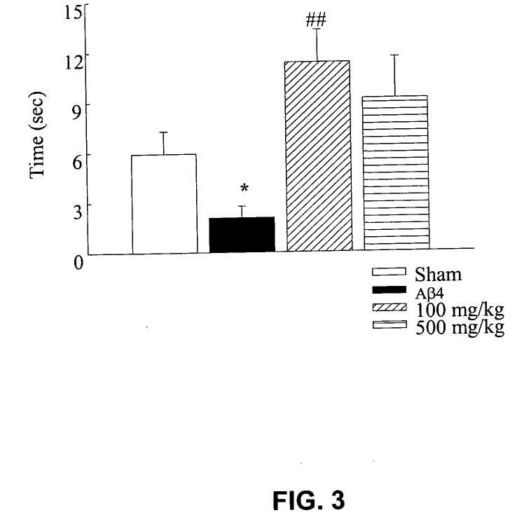

The results are shown in FIGS. 2 and 3. Polygonum multiflorum with a dosage of 100 mg/kg significantly improves the spatial working and reference memory. Besides, polygonum multiflorum with a dosage of 500 mg/kg also improves the spatial working and reference memory.

### EXAMPLE 5

### Water Extract of Polygonum Multiflorum for Treating APP Gene Transformed Mice

The mouse utilized in the example was Tg2576 (Hsiao et al., Science 1996), a human APP gene Swedish mutation transgenic mouse and also the first transgenic one. Amyloid loading and cognitive behavior are both observed in the mouse and it is regarded as a suitable animal model of Alzheimer disease.

Tg2576 mice aged 6 to 7 months were divided into two groups. The water extract of polygonum multiflorum and solvent were added into the drinking water of the experiment and control groups, respectively. Every month, the mice were subjected to Morris water maze test as described in Example 4 for one week.

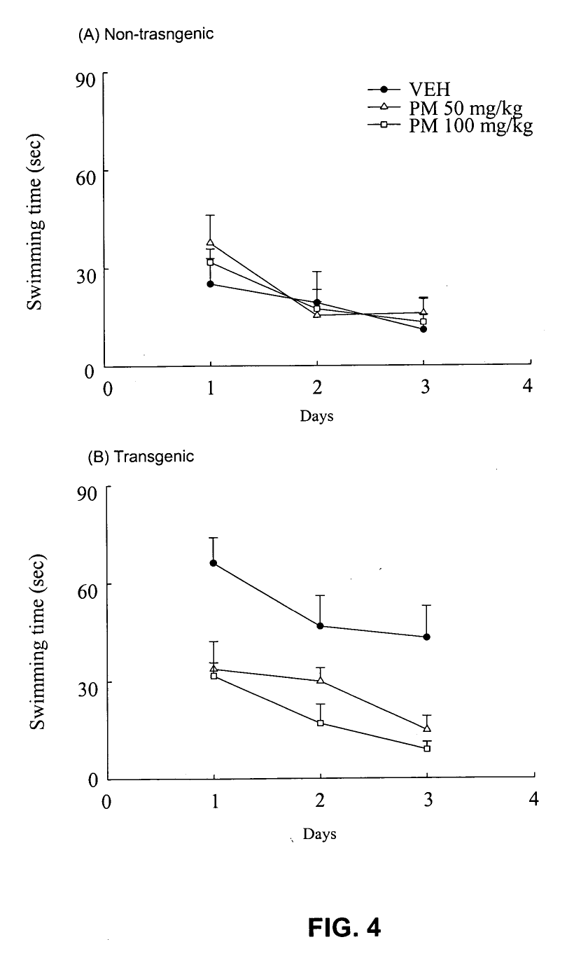

As shown in FIG. 4, the water extract of polygonum multiflorum with dosages of 50 to 100 mg/kg improves spatial working of water maze in the animal model. Additionally, the effect strengthens as the treating period increases.

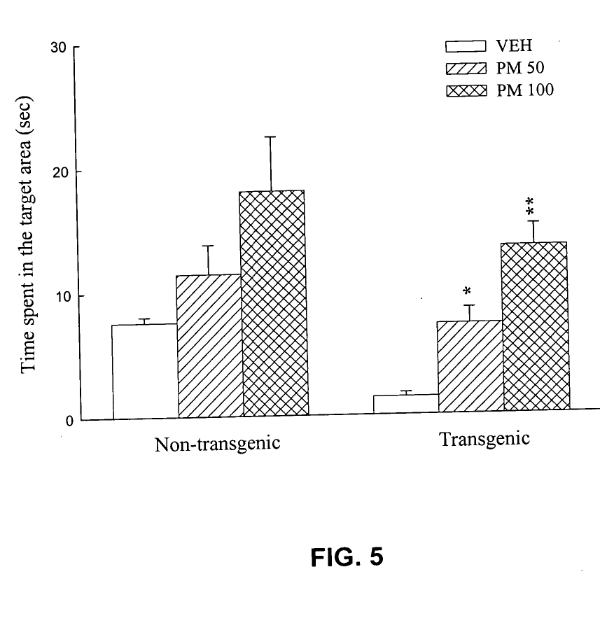

As shown in FIG. 5, the water extract of polygonum multiflorum improves reference memory in the animal model also. The effect seems to increase as the treating period increases.

Given the above, the learning ability of non-transgenic mice shows no significant difference between the experiment and control groups. However, the long-term treatment to the transgenic mice (Tg2576) improves the learning ability in the experiment group.

Alternatively, Tg2576 mice aged 15.5 months were divided into two groups. The water extract of polygonum multiflorum and solvent were directly fed to the experiment and control groups, respectively. The mice were subjected to Morris water maze test as described in Example 4 every month. The tests were terminated after significant difference between the experiment and control groups appeared.

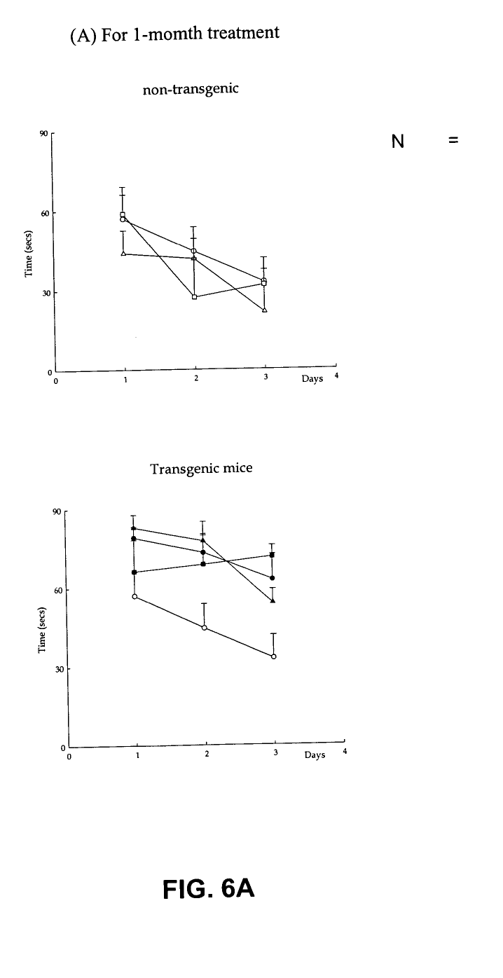

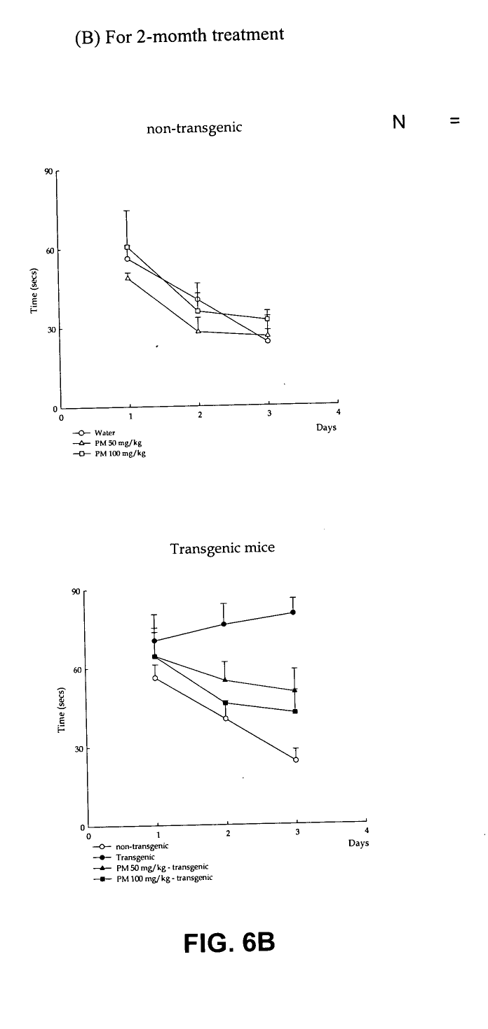

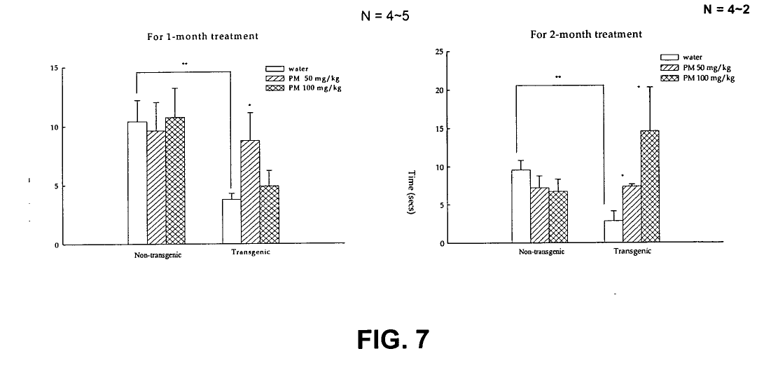

As shown in FIGS. 6 and 7, the water extract of polygonum multiflorum improves spatial reference memory in the animal model after one-month treatment. When the treatment elongated to two months, water extract of polygonum multiflorum with dosages of 50 and 100 mg/kg improves spatial working and reference memory significantly.

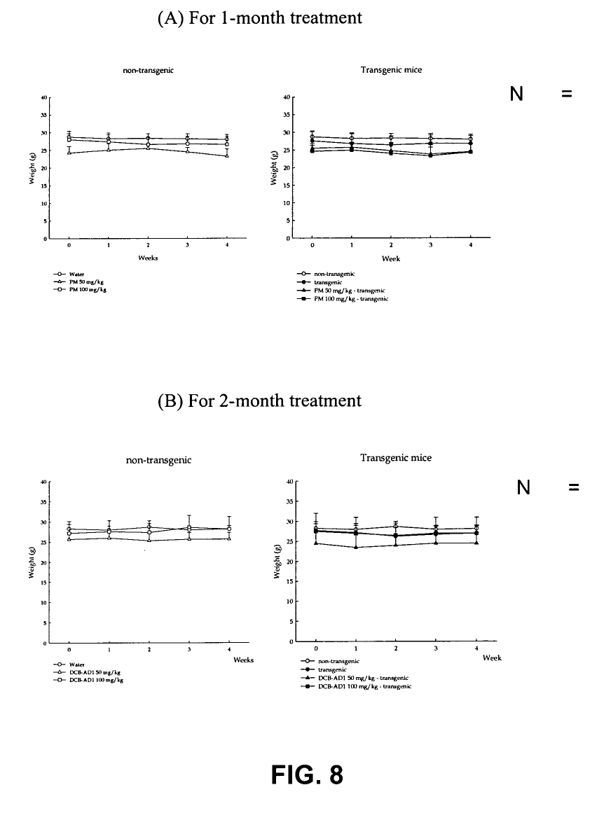

The body weight of the mice was also estimated and shown in FIG. 8. It evidences that the dosage and duration of treatment of water extract of polygonum multiflorum change the body weight of the mice very slightly.

While embodiments of the present invention have been illustrated and described, various modifications and improvements can be made by persons skilled in the art. It is intended that the present invention is not limited to the particular forms as illustrated, and that all the modifications not departing from the spirit and scope of the present invention are within the scope as defined in the appended claims.

What is claimed is:

1. A composition for treating amyloidoses and/or oxidation damage in nervous system comprising a therapeutically effective amount of the water extract of polygonum multiflorum.

2. The composition according to claim 1, wherein the amyloidoses comprises Alzheimer disease, stroke, dementia, prion diseases, spongiform encephalopathies, Kennedy disease, Pick disease, Parkinson disease, spinocerebellar ataxia, and Machado-Joseph disease.

3. The composition according to claim 1, wherein the amyloidoses comprises Alzheimer disease.

4. The composition according to claim 1, which is for inhibiting neuronal damage resulting from the amyloid β peptide aggregation.

5. The composition according to claim 4, which is for inhibiting neuronal damage resulting from the amyloid β 40 or 42 protein aggregation.

6. The composition according to claim 1, wherein the water extract is prepared by extracting polygonum multiflorum with water at the temperature of from 60 to 70° C.

7. The composition according to claim 1, wherein polygonum multiflorum is boiled with a black bean liquid and/or yellow rice wine.

8. The composition according to claim 1, which is in the form of powder, tablet, capsule, solution, tonic, or food composition.

9. A method for treating amyloidoses and/or oxidation damage in nervous system of a subject comprising administrating the subject a therapeutically effective amount of the water extract of polygonum multiflorum.

10. The method according to claim 9, wherein the amyloidoses comprises Alzheimer disease, stroke, dementia, prion diseases, spongiform encephalopathies, Kennedy disease, Pick disease, Parkinson disease, spinocerebellar ataxia, and Machado-Joseph disease.

11. The method according to claim 9, wherein the amyloidoses comprises Alzheimer disease.

12. The method according to claim 9, which is for inhibiting neuronal damage resulting from the amyloid β peptide aggregation.

13. The method according to claim 12, which is for inhibiting neuronal damage resulting from the amyloid β 40 or 42 protein aggregation.

14. The method according to claim 9, wherein the water extract is prepared by extracting polygonum multiflorum with water at the temperature of from 60 to 70° C.

15. The method according to claim 9, wherein polygonum multiflorum is boiled with a black bean liquid and/or yellow rice wine.
